(19) United States
(12) Patent Application Publication
(10) Pub. No.: US 2026/0187793 A1
(43) Pub. Date: Jul. 2, 2026

(54) METHOD, COMPUTER PROGRAM, AND COMPUTING DEVICE FOR GENERATING TRAINING DATASET BASED ON ENDOSCOPIC IMAGES

(71) Applicant: MedInTech Inc., Seoul (KR)

(72) Inventors: Myungjoon KIM, Gwacheon (KR); Seokho CHO, Seoul (KR); Donghyeok KIM, Seoul (KR)

(21) Appl. No.: 19/394,970

(22) Filed: Nov. 20, 2025

(30) Foreign Application Priority Data
Dec. 30, 2024 (KR) 10-2024-0200502

(51) Int. Cl. G06T 7/00; G06V 10/774; G06V 10/776; G16H 30/40

(52) U.S. Cl. CPC G06T 7/0012; G06V 10/774; G06V 10/776; G16H 30/40; G06T 2207/10068; G06T 2207/30096; G06V 2201/07

(57) ABSTRACT

According to an aspect of the present disclosure, there is provided a method of generating a training dataset based on endoscopic images. The method includes: inputting raw data to a plurality of models each trained to infer valid information for an endoscopic image acquired by capturing the inside of the body; generating final valid information for the raw data based on valid information output from each of the models; and generating a training dataset including the raw data and the final valid information. The final valid information is information required for training a model for analyzing the endoscopic image by using the training dataset, and includes a label for the raw data or additional information for the label.

### CROSS-REFERENCE TO RELATED APPLICATION

This application claims the benefit of Korean Patent Application No. 10-2024-0200502 filed on Dec. 30, 2024, which is hereby incorporated by reference herein in its entirety.

### BACKGROUND

### 1. Technical Field

The present disclosure relates to technology for generating a training dataset, and more specifically, to a method, computer program, and computing device for generating a training dataset based on endoscopic images.

### 2. Description of the Related Art

An endoscope is a general name for a medical device that allows a scope to be inserted into the body and internal organs to be observed without surgery or an autopsy. An endoscope inserts a scope into the body of an animal or human, radiates light, and visualizes the light reflected from the surface of an internal wall. Such endoscopes are categorized by purpose and body part. They may be basically categorized into rigid endoscopes, which have metal tubes, and flexible endoscopes, which are represented by digestive endoscopes.

Meanwhile, various artificial neural network models are being utilized in the field of endoscopy to improve the accuracy and efficiency of examinations. As an example, artificial neural network models are being utilized to detect polyps in real time during colonoscopy, classify them as benign or malignant, and/or automatically detect early cancerous lesions. Furthermore, artificial neural network models are being utilized to improve image quality by converting low-resolution endoscopic images into high-resolution images or removing various types of noise from the images.

In order to utilize an artificial neural network model, it first needs to be trained. That is, the process of preparing training data needs to be preceded. In particular, in the medical field such as the field of endoscopy, high-quality training data is crucial for ensuring the accuracy and reliability of a model.

However, preparing high-quality training data in the medical field presents various challenges. Medical data contains sensitive information such as patient health status and treatment records, thus making the process of collecting data complicated. Furthermore, artificial neural network models need to be trained on data from diverse patient populations, so that it is necessary to obtain data having diverse characteristics such as gender, age, and medical history. Therefore, there is required technology for preparing training data in large quantities while ensuring accuracy in the medical field, particularly in the field of endoscopy.

### SUMMARY

The present disclosure is intended to overcome the above-described problems of the related art, and is directed to a method, computer program, and computing device for generating a training dataset based on endoscopic images that generate a training dataset by generating labels or additional information for the labels by using the raw data of endoscopic images.

However, objects to be accomplished by the present disclosure are not limited to the object described above, and there may be other objects.

According to an aspect of the present disclosure, there is provided a method of generating a training dataset based on endoscopic images. The method includes: inputting raw data to a plurality of models each trained to infer valid information for an endoscopic image acquired by capturing the inside of the body; generating final valid information for the raw data based on valid information output from each of the models; and generating a training dataset including the raw data and the final valid information. The final valid information is information required for training a model for analyzing the endoscopic image by using the training dataset, and includes a label for the raw data or additional information for the label.

Alternatively, the model for analyzing the endoscopic image may include a model for detecting a target in the endoscopic image, the label may include whether the raw data contains the target, and the additional information for the label may include information about a confidence level for the label or the location of the target.

Alternatively, the target may include at least one of a lesion, a body fluid, a foreign substance, an endoscopic instrument, or a preset region of interest.

Alternatively, the model for analyzing the endoscopic image may include a model that predicts an operation to be performed by an endoscopic device based on the endoscopic image, the label may include the type of operation, and the additional information for the label may include a confidence level for the label.

Alternatively, the label may include a class representing an operation of the scope of the endoscopic device, including at least one of the up, down, left, right, forward, or backward operations of the scope.

Alternatively, the label may include a class representing an operation to be performed by at least one of the air pump, suction pump, or water pump of the endoscopic device.

Alternatively, the plurality of models may include first and second models that detect a target included in the raw data and output information about a region containing the target.

Alternatively, generating the final valid information may include: computing a score and a third region based on a first region and confidence level for the first region output from the first model and a second region and confidence level for the second region output from the second model; and generating the final valid information based on the third region according to the result of the comparison between the score and a reference value.

Alternatively, the plurality of models may include a first model that detects a target included in the raw data and outputs information about a region containing the target, and a second model that outputs whether the raw data contains a target.

Alternatively, generating the final valid information may include generating the final valid information based on a first region and confidence level for the first region output from the first model and a class and confidence level for the class output from the second model.

Alternatively, the plurality of models may include a first model that detects a target included in the raw data and outputs information about a region containing the target, a second model that outputs whether the raw data contains a target, and a third model that outputs a class for each of pixels constituting the raw data.

Alternatively, generating the final valid information may include generating the final valid information based on a first region and confidence level for the first region output from the first model, a class and confidence level for the class output from the second model, and a pixel-wise class output from the third model.

Alternatively, generating the final valid information may include comparing the distribution value of the pixel-wise class within the first region with a first reference value, comparing the confidence level for the class with a second reference value, and generating the final valid information based on the results of the respective comparisons.

According to another aspect of the present disclosure, there is provided a computer program stored in a computer-readable storage medium, the computer program causing operations for generating a training dataset based on endoscopic images to be performed when executed by at least one processor, wherein the operations include operations of: inputting raw data to a plurality of models each trained to infer valid information for an endoscopic image acquired by capturing the inside of the body; generating final valid information for the raw data based on valid information output from each of the models; and generating a training dataset including the raw data and the final valid information; wherein the final valid information is information required for training a model for analyzing the endoscopic image by using the training dataset, and includes a label for the raw data or additional information for the label.

According to another aspect of the present disclosure, there is provided a computing device for generating a training dataset based on endoscopic images, the computing device including: memory configured to store raw data for an endoscopic image acquired by capturing the inside of the body; and a processor configured to input raw data to a plurality of models each trained to infer valid information for an endoscopic image, to generate final valid information for the raw data by combining results output from the respective models, and to generate a training dataset including the raw data and the final valid information; wherein the final valid information is information required for training a model for analyzing the endoscopic image by using the training dataset, and includes a label for the raw data or additional information for the label.

According to at least one embodiment of the present disclosure, a plurality of neural network models that output different characteristics may be used to output valid information for a lesion and/or the like contained in an endoscopic image and each of the models reflects this valid information therein to generate label information for the endoscopic image by using final valid information. Accordingly, the accuracy of generated labels may be improved, and high-quality training data may be easily generated in large quantities.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a block diagram of an endoscopic system including an endoscopic device and a computing device according to one embodiment of the present disclosure;

FIG. 2 is a schematic diagram showing an artificial neural network model according to one embodiment of the present disclosure;

FIG. 3 is an exemplary diagram of an artificial neural network model according to one embodiment of the present disclosure;

FIG. 4 is a diagram illustrating the operation of computing final valid information according to one embodiment of the present disclosure;

FIGS. 5 and 6 are exemplary diagrams of artificial neural network models according to one embodiment of the present disclosure; and

FIG. 7 is a flowchart showing the operation of a computing device according to one embodiment of the present disclosure.

### DETAILED DESCRIPTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

The term “image” used herein may refer to multidimensional data composed of discrete image elements. In other words, the term “image” may be understood as a term that refers to a digital representation of an object visible to the human eye. For example, the term “image” may refer to multidimensional data composed of elements corresponding to pixels in a two-dimensional image. Alternatively, the term “image” may refer to multidimensional data composed of elements corresponding to voxels in a three-dimensional image.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

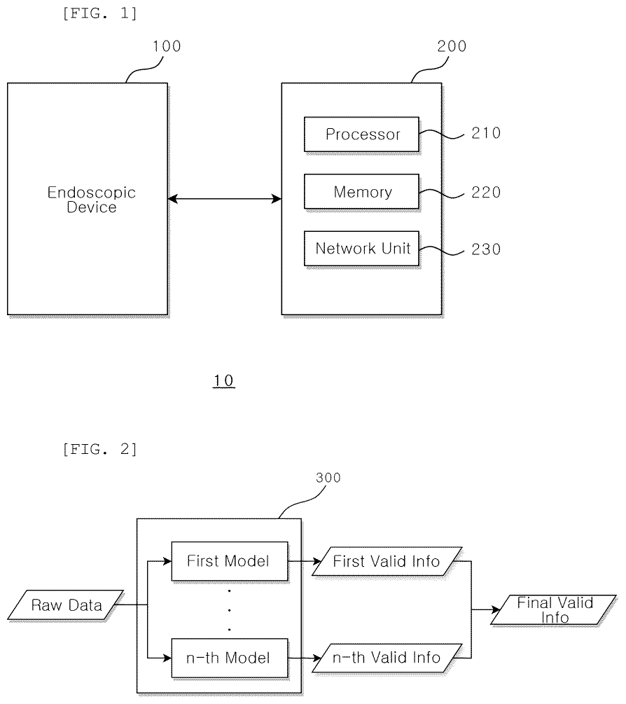

FIG. 1 is a block diagram of an endoscopic system including an endoscopic device and a computing device according to one embodiment of the present disclosure.

Referring to FIG. 1, an endoscopic system 10 includes an endoscopic device 100 configured to obtain various types of information, including medical images of the inside of the body, and a computing device 200 configured to receive the information about the inside of the body from the endoscopic device 100 and analyze it to restore the medical images of the inside of the body in three dimensions.

In the present specification, the endoscopic device 100 may be a rigid endoscope or a flexible endoscope. The rigid endoscope may include a straight tube made of high-strength metal or plastic, and the flexible endoscope may include a tube made of flexible material. Such a tube may be referred to as a scope. The scope of each of the rigid and flexible endoscopes may include an optical fiber or lens system for transmitting light and images therein. Furthermore, a channel for spraying air or water, a channel for inserting a biopsy instrument, and/or the like may be provided. The operations and configurations described in the present disclosure may be intended to control a rigid endoscope or a flexible endoscope. However, the type of endoscopic device 100 to which the present disclosure is applied is not limited thereto.

Furthermore, in the present specification, the endoscopic device 100 may be used for an animal or a human. That is, the “body” described herein may refer to either the body of an animal or the body of a human. The configuration of the endoscopic device 100 may be appropriately modified by taking into consideration the size of the body of an animal or a human and anatomical characteristics, and the operations and configurations described herein may also be appropriately modified according to the characteristics of the body.

The endoscopic device 100 may include a component capable of acquiring a medical image of the inside of a digestive organ, and a component capable of, if necessary, inserting an instrument to perform treatment or a procedure while viewing the medical image.

The endoscopic device 100 may include an output unit, a control unit, a driving unit, a pump unit, and a scope, and may further include a light source unit. The endoscopic device 100 may further include a network device that transmits and receives data to and from an external computing device 200, another endoscopic device 100, a hospital server, or the like.

The output unit may include a display that displays medical images. The output unit may include a display module capable of outputting visualized information or implementing a touch screen, such as a liquid crystal display (LCD), a thin film transistor-liquid crystal display (TFT LCD), an organic light-emitting diode (OLED) display, a flexible display, or a three-dimensional (3D) display.

The output unit may include various means for providing medical images or information about medical images. The output unit may display the medical images acquired via the scope or processed by the control unit. In addition to visual means, the output unit may provide information through auditory means. For example, the output unit may include a speaker that audibly provides alarms for medical images. Meanwhile, although one output unit is shown in FIG. 2, a plurality of output units may be provided. In this case, an output unit for displaying medical images acquired via the scope and an output unit for displaying information processed by the control unit may be separated from each other.

The control unit may control the overall operation of the endoscopic device 100. For example, the control unit may perform the operation of capturing a medical image through the scope, the operation of processing an acquired medical image, a control operations for performing a medical operation such as the spraying of cleaning water or suctioning, and a series of operations for controlling the movement of the scope. The control unit may include any type of device capable of processing data. According to an exemplary embodiment, the control unit may be a hardware-embedded data processing device having a physically structured circuit to perform functions expressed in code or instructions included in a program. Examples of the hardware-embedded data processing device may include processing devices such as a microprocessor, a central processing unit (CPU), a processor core, a multiprocessor, an application-specific integrated circuit (ASIC), or a field programmable gate array (FPGA), but the technical concept of the present disclosure is not limited thereto.

The control unit may control the movement of the scope through the driving unit connected to the scope. That is, the control unit may generate control signals to be provided to the driving unit to control the movement of the scope.

As an example, a series of operations in which the endoscopic device 100 of the present disclosure controls the scope may be performed as follows. A user may input the degree or direction of curvature of the scope through a manipulation unit. The input information is transmitted to the control unit, and the control unit may generate a signal to be provided to the driving unit by processing the input information. For example, the control unit may compute the locations, angles, angular velocities, and/or the like of the motors corresponding to the degree or direction of curvature set by the user, and may provide the information to the driving unit. The driving unit may generate power based on the signal from the control unit and transmit it to the scope. Accordingly, the scope may be moved or bent in response to the value input by the user.

The driving unit may provide the power required for the scope to be inserted into the body, to be bent within the body, and to be moved within the body. For example, the driving unit may include motors connected to wires within the scope, and a tension adjustment unit configured to adjust the tension of the wires.

The driving unit may control the scope in various directions by controlling the power of the motors. For example, the motors may include a plurality of motors that correspond to the directions in which an insertion portion at an end of the scope needs to be bent. Alternatively, the motors may include a plurality of motors that correspond to wires within the scope. More specifically, the driving unit may include a first motor configured to determine the x-axis movement of the scope, and a second motor configured to determine the y-axis movement of the scope. The x-axis location, y-axis location, z-axis location, and roll, pitch, and yaw values of the end of the scope may be determined based on the control of the driving unit, but the configuration of the driving unit is not limited thereto.

The tension adjustment unit may generate tension by receiving power from the motors and pulling the wires inside the scope. This allows the scope to be bent. The tension adjustment unit may adjust the tension applied to the plurality of wires inside the scope so that the scope can be bent according to the determined amount and direction of curvature.

The pump unit may include at least one of an air pump configured to inject air into the body through the scope, a suction pump configured to provide negative pressure or vacuum to suck air from the body through the scope, or a water pump configured to inject cleaning water into the body through the scope. Each of the pumps may include a valve for controlling the flow of fluid. The pump unit may be selectively opened and closed by the control unit. At least one of the suction pump, the water pump, or the air pump may be selectively opened and closed based on a control signal from the computing device 200 or the control of the control unit.

The scope may include an insertion part configured to be inserted into a digestive organ, and a manipulation part configured to control the movement of the insertion part and receive input from the user to perform various operations.

The insertion part is configured to be flexible, and is connected to the driving unit at one end thereof, so that the degree and direction of curvature can be determined by the driving unit. Since medical imaging and procedures are performed at the end of the insertion part, the scope may include pluralities of cables and tubes that extend to the end of the insertion part. The scope may include a light source lens, an objective lens, a working channel, and an air/water channel. An instrument for treating or manipulating a lesion during an endoscopic procedure may be inserted through the working channel. Air may be injected and cleaning water may be supplied through the air/water channel. Meanwhile, although the air/water channel is shown as the channel through which cleaning water is supplied in FIG. 2, it is not limited thereto. For example, a separate water jet channel (not shown) may be provided within the scope, and cleaning water may also be supplied through the water jet channel.

Meanwhile, the expression “the scope is bent by the control unit or driving unit” described herein may refer to a case where at least a portion, such as the insertion part, of the scope is bent.

The manipulation unit may include a plurality of input buttons that allow an endoscopist to control the steering of the insertion part and provide various functions (such as the capturing of an image, and the spraying of cleaning water) to perform procedures through the working channel and the air/water channel. For example, the manipulation unit may include a plurality of buttons or a joystick-type input device that indicates the direction of the scope.

The light source unit may include a light source that radiates light into the body through the endoscopic scope. The light source unit may include a lighting device that generates white light, or may include a plurality of lighting devices that generate light with different wavelength bands. The type of light source, the intensity of light, white balance, and/or the like may be set through the light source unit. Meanwhile, the above-described settings may also be set through the control unit. The light generated by the light source unit may be transmitted to the scope through a path such as an optical fiber.

The computing device 200 according to one embodiment of the present disclosure may be a hardware device that performs comprehensive data processing and computational operations or a part of the hardware device, or may be a software-based computing environment that is connected over a communication network. For example, the computing device 200 may be a server that is a subject that performs intensive data processing functions and shares resources, or may be a client that shares resources through interaction with a server. Furthermore, the computing device 200 may be a cloud system that enables pluralities of servers and clients to interact and comprehensively process data. The above description is merely one example of the type of computing device 200. Accordingly, the type of computing device 200 may vary within a range understandable to those skilled in the art based on the content of the present disclosure. The above description is merely one example of the type of computing device 200. Accordingly, the type of computing device 200 may be configured in various manners within a range understandable to those skilled in the art based on the content of the present disclosure.

Referring to FIG. 1, the computing device 200 according to one embodiment of the present disclosure may include a processor 210, memory 220, and a network unit 230. However, FIG. 1 is merely an example, and the computing device 200 may include other components for implementing a computing environment. Furthermore, only some of the components disclosed above may be included in the computing device 200.

The processor 210 according to one embodiment of the present disclosure may be understood as a constituent unit including hardware and/or software for performing computing operations. For example, the processor 210 may read a computer program and perform data processing for machine learning. The processor 210 may process operation processes such as the processing of input data for machine learning, the extraction of features for machine learning, and the computation of errors based on backpropagation. The processor 210 for performing such data processing may include a central processing unit (CPU), a general purpose graphics processing unit (GPGPU), a tensor processing unit (TPU), an application specific integrated circuit (ASIC), and a field programmable gate array (FPGA). Since the types of processor 210 described above are only examples, the type of processor 210 may be configured in various manners within a range understandable to those skilled in the art based on the content of the present disclosure.

The processor 210 according to the present disclosure may extract valid information from endoscopic images acquired via the endoscopic device 100, and may generate a training dataset containing the endoscopic images and the valid information. A neural network model may be trained using the training dataset. The neural network model may be a model for analyzing endoscopic images. The endoscopic device may be controlled based on the analysis results of the endoscopic images output by the neural network model. For example, the configuration of the endoscopic device may be physically controlled based on the analysis results, or other control information that influences the operation of the endoscopic device may be generated. As an example, the neural network model may identify a region containing a target, such as a lesion, from an endoscopic image, or may perform the operation of determining whether the endoscopic image is normal or is an image containing a target.

In the present specification, the term “target” may include at least one of a lesion which occurs inside the body, a region where a foreign substance such as body fluid or blood is present, and a pre-set region of interest. For example, the target may include a region where blood is distributed, or may include a region where blood vessels are concentrated. Alternatively, the target may include a region where a focus is blurred. Alternatively, the target may include a region where a surgical instrument is shown. Although a case where the target is a lesion is described as an example in the following, this is merely an example and the type of target is not limited thereto.

In the present specification, the term “valid information” may include information used as ground truth by the neural network model that is trained using a training dataset. The information used as ground truth may include labels for endoscopic images or additional information about the labels. For example, when the neural network model is trained to identify a target, such as a region containing a lesion, the valid information may include coordinate information for the region containing the lesion. Alternatively, the valid information may include pixel-wise class information for segmenting a region corresponding to the lesion in an endoscopic image. For example, when the neural network model is trained to classify input endoscopic images into normal images and images containing lesions, the valid information may include tags or label information for the endoscopic images.

The operation of the neural network model described above is exemplary, and any neural network model that generates information for analyzing endoscopic images is usable. For example, the neural network model may be trained to generate information for controlling the pump of the endoscopic device 100 based on endoscopic images. In this case, the valid information may include labels indicating the class of operations to be performed by at least one of the air pump, suction pump, or water pump of the endoscopic device 100.

Alternatively, the neural network model may be trained to generate information that controls the operation of the scope of the endoscopic device 100 based on endoscopic images. In this case, the valid information may include labels indicating a class for at least one of the up, down, left, right, forward, or backward operations of the scope.

The processor 210 may utilize a plurality of neural network models to extract valid information from endoscopic images, and may generate final valid information based on the pieces of information output from the respective neural network models. The plurality of neural network models may all perform the same function, or may include at least one neural network model that performs a different function.

The processor 210 may train each of a plurality of neural network models that output valid information. In this case, each of the neural network models may include at least one neural network. The neural network may include, but is not limited to, network models such as a Deep Neural Network (DNN), a Recurrent Neural Network (RNN), a Bidirectional Recurrent Deep Neural Network (BRDNN), a Multilayer Perceptron (MLP), a Convolutional Neural Network (CNN), or a Transformer.

That is, according to the present disclosure, a training dataset required for training a neural network model may be generated using only raw data without labels or additional label information for the labels. Furthermore, the time and effort required for the task of labeling correct answer information may be reduced, and thus a large, high-quality training dataset may rapidly be acquired. Furthermore, final valid information is generated by combining the outputs of the plurality of neural network models to generate a single training dataset, thereby improving the accuracy of training data.

The memory 220 according to one embodiment of the present disclosure may be understood as a constituent unit including hardware and/or software for storing and managing data that is processed in the computing device 200. That is, the memory 220 may store any type of data generated or determined by the processor 210 and any type of data received by the network unit 230. For example, the memory 220 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, or an optical disk. Furthermore, the memory 220 may include a database system that controls and manages data in a predetermined system. Since the types of memory 220 described above are only examples, the type of memory 220 may be configured in various manners within a range understandable to those skilled in the art based on the content of the present disclosure.

The memory 220 may structure, organize, and manage the data required for the processor 210 to perform operations, data combinations, and program code executable by the processor 210. Furthermore, the memory 220 may store program codes that cause the processor 210 to generate training data.

The memory 220 according to the present disclosure may store raw data for endoscopic images acquired via the endoscopic device 100, i.e., endoscopic images acquired by capturing the inside of the body. Furthermore, the memory 220 may store parameter information included in the plurality of neural network models, a plurality of pieces of valid information output from the respective neural network models, and reference values used to output final valid information from the plurality of pieces of valid information.

The network unit 230 according to one embodiment of the present disclosure may be understood as a constituent unit that transmits and receives data via any type of known wired/wireless communication system. For example, the network unit 230 may perform data transmission and reception by using a wired/wireless communication system such as a local area network (LAN), a wideband code division multiple access (WCDMA) network, a long term evolution (LTE) network, the wireless broadband Internet (WiBro), a 5th generation mobile communication (5G) network, an ultra-wideband wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 230 may be applied in various manners other than the above-described examples.

The network unit 230 may receive data, required for the processor 210 to perform operations, through wired/wireless communication with any system, any client, or the like. Furthermore, the network unit 230 may transmit data, generated through the operations of the processor 210, through wired/wireless communication with any system, any client, or the like. For example, the network unit 230 may receive basic data, including a captured image, through communication with a picture archiving and communication system (PACS), a cloud server that performs tasks such as the standardization of medical data, the endoscopic device 100, or the like. The network unit 230 may transmit various types of data generated via the operations of the processor 210 through communication with the above-described system, server, or endoscopic device 100, or the like.

Meanwhile, although the endoscopic device 100 and the computing device 200 are shown as separate in FIG. 1, this is merely exemplary. The computing device 200 may be provided within the endoscopic device 100 and constitute a part of the endoscopic device 100.

The network unit 230 according to the present disclosure may transmit the valid information, the final valid information, or the training dataset generated by the processor 210 out of the computing device 200. As an example, the training dataset may be transmitted to an external computing device 200 that trains a neural network model controlling the endoscopic device 100 based on the training dataset generated by the processor 210.

FIG. 2 is a schematic diagram illustrating an artificial neural network model according to one embodiment of the present disclosure.

Referring to FIG. 2, an artificial neural network model 300 may identify a detection target contained in raw data, and may output information about the target as final valid information. In this case, the detection target may refer to a target. The raw data refers to an image acquired via the endoscopic device 100, and may not contain any information about the target. The target may be a lesion, a foreign substance, an endoscopic instrument, a specific region, a specific part, or the like.

When the target is a lesion, the final valid information may include a bounding box containing the lesion, class information for pixels corresponding to the lesion, a label indicating the image containing the lesion, a confidence level for the lesion, the type of lesion, the size of the lesion, and/or the like.

That is, the artificial neural network model 300 may receive raw data, and may use a plurality of pieces of valid information, which are the results output from a plurality of models, to generate label information corresponding to the raw data. That is, information for utilizing the raw data as training data may be generated. In this case, the artificial neural network model 300 according to the present disclosure may provide a method of carefully generating training data by undergoing determination steps for ensuring reliability without directly using the results output from the plurality of models that analyze various characteristics in raw data.

The artificial neural network model 300 may include a plurality of models, for example, first to n-th models. Each of the first to n-th models may be a trained model, and, as an example, may be trained by the computing device 200.

As an example, the computing device 200 may train the first to n-th models to output valid information from endoscopic images by matching endoscopic images and labels for the endoscopic images and inputting matching results to the first to n-th models. This example is an embodiment in which the first to n-th models perform training through supervised learning. The first to n-th models may be trained to output labels or additional information about the labels from endoscopic images through various learning methods, such as semi-supervised learning, unsupervised learning, reinforcement learning, and self-supervised learning.

In this case, the first to n-th models may be trained in the same manner or in different manners. Furthermore, the first to n-th models may be trained to output the same type of valid information or different types of valid information.

Each of the trained first to n-th models may receive raw data for endoscopic images, may analyze the images contained in the endoscopic images, and may output valid information. In this case, the types of the first to n-th models and the parameters constituting the individual models may differ from each other. Accordingly, even when the first to n-th models have received a single set of raw data, first to n-th valid information output may differ from each other. Therefore, the computing device 200 may generate final valid information based on the first to n-th valid information to generate labels for the raw data or additional information for the labels.

In this case, when the types of the first to n-th models are the same, the formats of the first to n-th valid information may be similar to each other. Accordingly, the computing device 200 may extract redundant information from the first to n-th valid information, and may compare the redundant information with a reference value to generate final valid information.

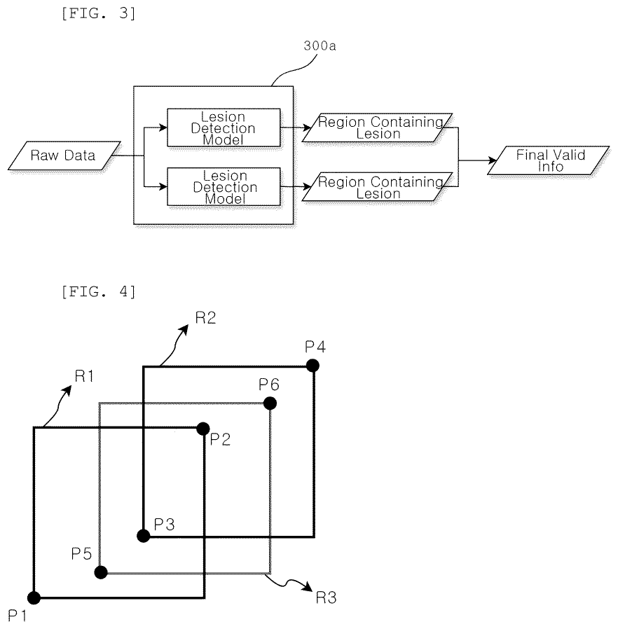

Meanwhile, when the types of first to n-th models are different, the formats of the first to n-th valid information may differ from each other. Accordingly, the computing device 200 may generate final valid information by using the values, included in the first to n-th valid information, as thresholds. Specific details will be described below with reference to FIGS. 3 to 6.

The computing device 200 may generate a training dataset by matching the final valid information and the raw data with each other. As an example, the final valid information may include the coordinate information of a bounding box containing a lesion, and the confidence level of this bounding box. The training dataset thus generated may be utilized as training data for the artificial neural network model 300 that detects lesions.

That is, according to the present disclosure, the computing device 200 may easily generate a large amount of training data by generating highly reliable label information using raw data.

Meanwhile, the types of models constituting the artificial neural network model 300 may be determined based on the characteristics of raw data, the characteristics of an artificial neural network model to be trained using the training dataset, and the operation of the artificial neural network model. That is, the type and amount of training data required may vary depending on the purpose of the artificial neural network model. To appropriately provide the training data required for the artificial neural network model, according to the present disclosure, an artificial neural network model that generates the label information required by the artificial neural network model may be selectively utilized. This allows for the efficient generation of training data.

As an example, depending on the purpose or role of the artificial neural network model, that is, a model for controlling an endoscopic device by using a training dataset, a configuration may be provided to select which of the plurality of artificial neural network models (300a, 300b, 300c) will be used to prepare a training dataset. Depending on the role of the endoscopic device control model, an appropriate one of the plurality of artificial neural network models 300a, 300b, and 300c may be recommended.

In the following, a case where the target is a lesion will be described as an example. As an example, when the artificial neural network model performs the operation of simply providing an alarm when a lesion occurs, the artificial neural network model 300 may be configured as a model optimized for determining the absence/presence and type of lesion. As an example, it may be the artificial neural network model 300b of FIG. 5.

As an example, when the artificial neural network model performs the operation of providing the location of a lesion, the artificial neural network model 300 may be configured as a model optimized for determining the location of a lesion. As an example, this may be the artificial neural network model 300a of FIG. 3. In this case, the artificial neural network model 300a uses a plurality of lesion detection models and takes into consideration consistency between the output results of the respective lesion detection models, making it suitable for providing highly reliable training data.

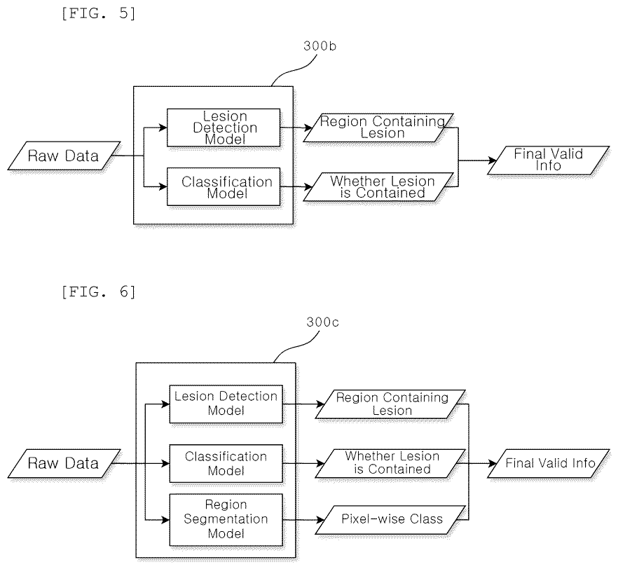

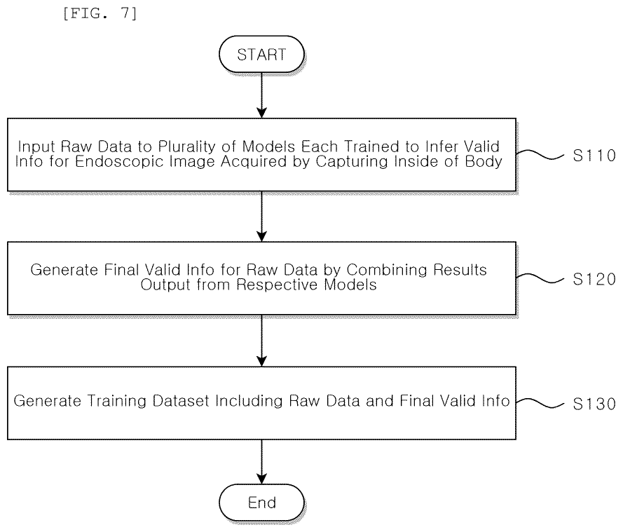

For example, when the primary purpose of the artificial neural network model is to provide the detailed location of a lesion, the artificial neural network model 300 may be configured as a model optimized for providing a region identified as a lesion. As an example, this may be the artificial neural network model 300c of FIG. 6.

For example, an appropriate one of a plurality of artificial neural network models 300a, 300b, and 300c may be recommended based on the priority considered in the endoscopic device control model. When it is important to precisely identify a region such as a lesion, a foreign substance, or the like, it may be recommended to generate training data by using the artificial neural network model 300c. When it is important to identify the type of lesion, it may be recommended to generate training data by using the artificial neural network model 300b. When it is important to reliably identify a lesion region, it may be recommended to generate training data by using the artificial neural network model 300a.

FIG. 3 is an exemplary diagram of an artificial neural network model according to one embodiment of the present disclosure, and FIG. 4 is a diagram illustrating the operation of computing final valid information according to one embodiment of the present disclosure.

Referring to FIG. 3, the artificial neural network model 300a corresponds to an embodiment of the artificial neural network model 300 of FIG. 2. The first to n-th models may all be models trained to output information about regions containing lesions by detecting the lesions. Accordingly, each of the lesion detection models may detect a lesion contained in raw data and output a region containing the lesion. That is, each of the lesion detection models may output the coordinates of a region containing a lesion, class information indicating the type of lesion, and the confidence level of an inference result.

In this case, the lesion detection model may output a region containing a lesion as a bounding box, a pixel-wise class, an attention map, a heat map, key point location information, or the like.

That is, the artificial neural network model 300a may use a plurality of lesion detection models that output lesion classification results and regions containing lesions. By doing this, final valid information may be generated based on consistency between the values output from the respective lesion detection models.

Referring to FIG. 4, the computing device 200 may generate final valid information based on the pieces of information output by respective lesion detection models.

As an example, a first lesion detection model may include a lesion called class 1 in raw data, may output a bounding box for the corresponding lesion as R1, and may output a first value conf1 as its confidence level. Furthermore, a second lesion detection model may include a lesion called class 1 in raw data, may output a bounding box for the corresponding lesion as R2, and may output a second value conf2 as its confidence level. In this case, when a case where the bounding box is a rectangle is taken as an example, R1 may be determined to be P1(x1, y1), P2(x2, y2), and R2 may be determined to be P3(x3, y3), P4(x4, y4). In this case, the coordinates of a bounding box R3 included in final valid information may be computed using Equation 1 below. The coordinates of R3 may be determined to be P5(x5, y5), P6(x6,y6).

$$x 5 = \frac{conf 1}{conf 1 + conf 2} \times x 1 + \frac{conf 2}{conf 1 + conf 2} \times x 3 \qquad ( 1 )$$

$$y 5 = \frac{conf 1}{conf 1 + conf 2} \times y 1 + \frac{conf 2}{conf 1 + conf 2} \times y 3$$

$$x 6 = \frac{conf 1}{conf 1 + conf 2} \times x 2 + \frac{conf 2}{conf 1 + conf 2} \times x 4$$

$$y 6 = \frac{conf 1}{conf 1 + conf 2} \times y 2 + \frac{conf 2}{conf 1 + conf 2} \times y 4$$

That is, the bounding box included in the final valid information may be generated by reflecting therein the bounding boxes and confidence levels output by the respective lesion detection models. Meanwhile, the shape of the bounding box does not necessarily have to be rectangular. When the shape is not rectangular, the bounding box to be reflected in the final valid information may be determined based on the coordinates and confidence levels of the points that determine the bounding box.

Meanwhile, the computing device 200 may determine whether to adopt the bounding box, generated in this manner, in the final valid information. As an example, the computing device 200 may compute a score based on each bounding box and its confidence level, and compare the score with a reference value to determine whether to adopt the bounding box. As an example, the computing device 200 may compute a score based on a first value representing the confidence level of bounding box R1, a second value representing the confidence level of bounding box R2, and a value representing the Intersection over Union (IoU) of bounding boxes R1 and R2. The computing device 200 may include bounding box R3 in the final valid information if the score is greater than or equal to a reference value.

Furthermore, when pieces of class information for the type of lesion output by the lesion detection models are different, the computing device 200 may not adopt the bounding box R3 as the final valid information. In this case, the raw data may be regarded as not containing a lesion.

FIGS. 5 and 6 are exemplary diagrams of artificial neural network models according to one embodiment of the present disclosure.

The artificial neural network model 300b of FIG. 5 and the artificial neural network model 300c of FIG. 6 correspond to embodiments of the artificial neural network model 300 of FIG. 2. In the following, descriptions similar to those of the artificial neural network model 300 of FIG. 3 given above will be omitted.

Referring to FIG. 5, the artificial neural network model 300b corresponds to one embodiment of the artificial neural network model 300 of FIG. 2. The artificial neural network model 300b may include a model trained to output information for a region containing a lesion by detecting the lesion from an endoscopic image, and may further include a model trained to determine whether a lesion is included in an endoscopic image or what type of lesion is included. In this case, the classification model may output a class indicating whether a lesion is present, class information indicating the type of lesion when present, and a confidence level for an inference result.

The computing device 200 may generate final valid information based on the bounding box and its confidence level output by the lesion detection model and the class and its confidence level output by the classification model.

As an example, the bounding box included in the final valid information may be output from the lesion detection model, and the class information included in the final valid information may be a class output from the classification model. The computing device 200 may compare the confidence level output from the classification model with a reference value, and may adopt the corresponding information as the final information when it is equal to or higher than the reference value.

In this case, the classification model may have a higher capability to infer the presence/absence of a lesion and the type of lesion than the lesion detection model. Accordingly, when the class information for a lesion output from the classification model differs from that output from the lesion detection model, the result output from the classification model may be used. That is, when both the classification model and the lesion detection model are used together, the classification model functions to compensate for the shortcomings of the lesion detection model.

Furthermore, when the classification model is trained using supervised learning, images and class information for lesions may be used as training data. When the lesion detection model is trained using supervised learning, images, class information for lesions, and bounding box information may be used as training data. Accordingly, the use of a classification model may reduce the difficulty of training and accelerate training speed.

That is, in a situation in which it is necessary to generate training data to be input to a model for accurately determining the type of lesion rather than accurately determining the location of the lesion, the artificial neural network model 300b of FIG. 5 may be more appropriate than other artificial neural network models. As an example, a model trained using the final valid information generated in FIG. 5 may be a model that performs endoscopic procedures, detects lesions in real time, and provides alarms. In this case, the most important consideration may be that the model, which has been trained on a large number of lesion cases, accurately identifies the types of lesions. Accordingly, to train a model that performs such operations, the use of the artificial neural network model 300b of FIG. 5 to provide training data may be most appropriate.

Meanwhile, although a single lesion detection model is shown in FIG. 5, there may be a plurality of lesion detection models. In this case, as in the method described in conjunction with FIGS. 3 and 4, a bounding box may be determined, and final valid information may be generated based on the coordinates and information of the determined bounding box and the information output from the classification model.

Referring to FIG. 6, the artificial neural network model 300c corresponds to one embodiment of the artificial neural network model 300 of FIG. 2. The artificial neural network model 300c may include a lesion detection model, a lesion classification model, and a region segmentation model. The region segmentation model may output a pixel-wise class constituting an endoscopic image. Accordingly, the region segmentation model may display a region corresponding to a target region, such as a lesion, a foreign substance, a bodily fluid, or the like, on the endoscopic image on a pixel-wise basis.

The computing device 200 may generate valid information based on the bounding box and its confidence level output from the lesion detection model, the class and its confidence level output from the classification model, and the pixel-wise class output from the region segmentation model.

As an example, the computing device 200 may determine the region in which the class output from the lesion detection model and the class output from the region segmentation model are identical in the region included in the bounding box. Furthermore, the computing device 200 may determine whether to include the bounding box in the final valid information based on the proportion of the corresponding region within the bounding box. Furthermore, whether to adopt the bounding box may be determined secondary based on the confidence level output from the classification model.

The artificial neural network model 300c utilizes the region segmentation model, thereby improving the accuracy of the bounding box compared to the artificial neural network model 300a of FIG. 3, which uses only the lesion detection model. That is, this reduces the possibility that a non-lesion region is included in the bounding box.

To train a region segmentation model by using supervised learning, images and pixel-wise class information may be used as training data. Accordingly, preparing training data may be more difficult than that for the classification model or lesion detection model, which may increase the difficulty of training. However, when it is determined that location information is more important than class information, inference accuracy is required even when the difficulty of training increases. Accordingly, it may be necessary the use an artificial neural network model 300c to generate final valid information.

As an example, a model trained using the final valid information generated in FIG. 6 may be a model that predicts the operations to be performed by an endoscopic device based on endoscope images. For example, the model may supply cleaning water or perform suction based on an endoscope image. In this case, the location information of the lesion to be sprayed with cleaning water and the location information of the target body fluid or foreign substance to be suctioned may be of the utmost importance. Accordingly, to train a model performing these operations, it may be most appropriate to use the artificial neural network model 300c of FIG. 6 to provide training data.

According to the present disclosure, a plurality of neural network models that output different characteristics from a single endoscopic image may be used to output valid information about a lesion and/or the like, and each of the models may reflect this valid information therein to generate label information for the endoscopic image by using final valid information. Accordingly, the accuracy of generated labels may be improved, and high-quality training data may be easily generated in large quantities.

FIG. 7 is a flowchart showing the operation of a computing device according to one embodiment of the present disclosure.

Referring to FIG. 7, the computing device 200 may input raw data to a plurality of models each trained to infer valid information for an endoscopic image acquired by capturing the inside of the body in step S110.

The computing device 200 may generate final valid information for the raw data based on the valid information output from each of the models in step S120. In this case, the final valid information may include information required for training a model for analyzing endoscopic images by using a training dataset. That is, it may include the label for raw data or additional information for the label. The result obtained by analyzing an endoscopic image may be used to physically control the configuration of the endoscopic device or to generate other control information that influences the operation of the endoscopic device.

In one embodiment, the model for analyzing endoscopic images may include a model for detecting targets in endoscopic images. In this case, the label may indicate whether the raw data contains a target. Furthermore, additional information for the label may include information for the reliability of the label or the location of the target. The target may include at least one of a lesion, a foreign substance, a bodily fluid, or a preset region of interest.

As an embodiment, the model for analyzing endoscopic images may include a model that predicts the operations to be performed by an endoscopic device based on endoscopic images. In this case, the label may include the type of operation. The additional information for the label may include a confidence level for the label. For example, the label may include a class representing the operation of the endoscopic device, including at least one of the up, down, left, right, forward, or backward operations of the scope. Alternatively, the label may include a class representing the operation to be performed by at least one of the air pump, suction pump, or water pump of the endoscopic device.

The computing device 200 may generate a training dataset including the raw data and the final valid information in step S130.

As an embodiment, the plurality of models may include first and second models that detect targets included in raw data and output information about regions containing the targets. In this case, in step S120, the computing device 200 may compute a score and a third region based on the first region and the confidence level for the first region output from the first model and the second region and the confidence level for the second region output from the second model. Furthermore, the computing device 200 may generate final valid information based on the third region based on the result of comparison between the score and a reference value.

As an embodiment, the plurality of models may include a first model that detects a target included in raw data and outputs information about a region containing the target, and a second model that outputs whether raw data contains a target. In step S120, the computing device 200 may generate final valid information based on the first region and the confidence level for the first region output from the first model and the class and the confidence level for the class output from the second model.

As an embodiment, the plurality of models may include a first model that detects a target in raw data and outputs information about a region containing the target, a second model that outputs whether raw data contains a target, and a third model that outputs a class for each of the pixels constituting raw data. In this case, in step S120, the computing device 200 may generate final valid information based on the first region and the confidence level for the first region output from the first model, the class and the confidence level for the class output from the second model, and the pixel-wise class output from the third model.

In addition, the computing device 200 may compare the distribution value of the pixel-wise class within the first region with a first reference value, may compare the reliability of the class with a second reference value, and may generate final valid information based on the results of the respective comparisons.

In this case, the above-described target may be, for example, a lesion, but may also include at least one of a body fluid, a foreign substance, an endoscopic instrument, a specific region, a specific part, or a defocused region, other than the lesion.

The above description of the present disclosure is intended for illustrative purposes only, and those skilled in the art will readily appreciate that modifications into other specific forms may be made without altering the technical spirit or essential features of the present disclosure. Therefore, the embodiments described above should be understood as illustrative and not restrictive in all respects. For example, components described as single may be implemented in a distributed form, and similarly, components described as distributed may be implemented in a combined form.

The scope of the present disclosure is defined by the attached claims rather than the above detailed description, and all changes or modifications derived from the meanings and scope of the claims and their equivalent concepts should be interpreted as being encompassed in the scope of the present disclosure.

What is claimed is:

1. A method of generating a training dataset based on endoscopic images, the method being performed by a computing device including at least one processor, the method comprising:
inputting raw data to a plurality of models each trained to infer valid information for an endoscopic image acquired by capturing an inside of a body;
generating final valid information for the raw data based on valid information output from each of the models; and
generating a training dataset including the raw data and the final valid information;
wherein the final valid information is information required for training a model for analyzing the endoscopic image by using the training dataset, and comprises a label for the raw data or additional information for the label.

2. The method of claim 1, wherein:
the model for analyzing the endoscopic image comprises a model for detecting a target in the endoscopic image;
the label comprises whether the raw data contains the target; and
the additional information for the label comprises information about a confidence level for the label or a location of the target.

3. The method of claim 2, wherein the target comprises at least one of a lesion, a body fluid, a foreign substance, an endoscopic instrument, or a preset region of interest.

4. The method of claim 1, wherein:
the model for analyzing the endoscopic image comprises a model that predicts an operation to be performed by an endoscopic device based on the endoscopic image;
the label comprises a type of operation; and
the additional information for the label comprises a confidence level for the label.

5. The method of claim 4, wherein the label comprises a class representing an operation of a scope of the endoscopic device, including at least one of up, down, left, right, forward, or backward operations of the scope.

6. The method of claim 4, wherein the label comprises a class representing an operation to be performed by at least one of an air pump, suction pump, or water pump of the endoscopic device.

7. The method of claim 1, wherein the plurality of models comprise first and second models that detect a target included in the raw data and output information about a region containing the target.

8. The method of claim 7, wherein generating the final valid information comprises:
computing a score and a third region based on a first region and confidence level for the first region output from the first model and a second region and confidence level for the second region output from the second model; and
generating the final valid information based on the third region according to a result of comparison between the score and a reference value.

9. The method of claim 1, wherein the plurality of models comprise a first model that detects a target included in the raw data and outputs information about a region containing the target, and a second model that outputs whether the raw data contains a target.

10. The method of claim 9, wherein generating the final valid information comprises generating the final valid information based on a first region and confidence level for the first region output from the first model and a class and confidence level for the class output from the second model.

11. The method of claim 1, wherein the plurality of models comprise a first model that detects a target included in the raw data and outputs information about a region containing the target, a second model that outputs whether the raw data contains a target, and a third model that outputs a class for each of pixels constituting the raw data.

12. The method of claim 11, wherein generating the final valid information comprises generating the final valid information based on a first region and confidence level for the first region output from the first model, a class and confidence level for the class output from the second model, and a pixel-wise class output from the third model.

13. The method of claim 12, wherein generating the final valid information comprises comparing a distribution value of the pixel-wise class within the first region with a first reference value, comparing the confidence level for the class with a second reference value, and generating the final valid information based on results of the respective comparisons.

14. A computer program stored in a computer-readable storage medium, the computer program causing operations for generating a training dataset based on endoscopic images to be performed when executed by at least one processor, wherein the operations comprise operations of:
inputting raw data to a plurality of models each trained to infer valid information for an endoscopic image acquired by capturing an inside of a body;
generating final valid information for the raw data based on valid information output from each of the models; and
generating a training dataset including the raw data and the final valid information;
wherein the final valid information is information required for training a model for analyzing the endoscopic image by using the training dataset, and comprises a label for the raw data or additional information for the label.

15. A computing device for generating a training dataset based on endoscopic images, the computing device comprising:
memory configured to store raw data for an endoscopic image acquired by capturing an inside of a body; and
a processor configured to input raw data to a plurality of models each trained to infer valid information for an endoscopic image, to generate final valid information for the raw data by combining results output from the respective models, and to generate a training dataset comprising the raw data and the final valid information;
wherein the final valid information is information required for training a model for analyzing the endoscopic image by using the training dataset, and comprises a label for the raw data or additional information for the label.
